(19) United States
(12) Patent Application Publication
(10) Pub. No.: US 2018/0255801 A1
(43) Pub. Date: Sep. 13, 2018

(54) COMPOSITIONS CONTAINING TEA, TEA-LIKE OR OTHER NON-TOBACCO LEAF-LIKE MATERIAL

(71) Applicant: Pocket Tea, LLC, Margaretville, NY (US)

(72) Inventor: Daniel J. Victor, Weston, CT (US)

(21) Appl. No.: 15/978,301

(22) Filed: May 14, 2018

Related U.S. Application Data
(63) Continuation-in-part of application No. 15/100,288, filed on May 27, 2016.
(60) Provisional application No. 62/506,075, filed on May 15, 2017.

(51) Int. Cl. A23F 3/34; A23L 33/105; A61K 36/185; A61K 9/00; B65D 85/804

(52) U.S. Cl. CPC A23F 3/34; A23L 33/105; A61K 36/185; A61K 9/009; B65D 85/8043; A23V 2250/214; A23V 2200/21; A23V 2250/056; A23V 2250/11; A23V 2250/1944; A23V 2002/00

(57) ABSTRACT

A non-tobacco formulation for oral administration in dry form includes a non-tobacco tea-like composition comprising plant material, an additive mixed with the composition and responsive to being wetted by a user's saliva to cause gaseous bubbles to be released and provide a popping or cracking sensation in the user's mouth, and a liquid-porous container (e.g., a pouch) having walls enclosing the composition and additive therein while permitting liquid to pass therethrough. Alternatively the additive may coat the pouch walls.

### CROSS REFERENCE TO RELATED APPLICATIONS

This application is a non-provisional application claiming priority from U.S. Provisional Application No. 62/506,075, entitled “Pouch Contained Tea and Tea-Like Compositions”, and filed May 15, 2017, the disclosure in which is incorporated in its entirety herein by this reference.

This application is a continuation-in-part application of and claims priority from commonly owned U.S. patent application Ser. No. 15/100,288, filed Jan. 30, 2015 as PCT/US15/13927 and entitled “Tea Composition For Oral Administration”, which claims priority from U.S. Provisional Application No. 61/965,474 filed on Jan. 31, 2014, the disclosures in which are incorporated herein in their entireties by this reference.

### BACKGROUND

### Field of the Disclosure

The present disclosure pertains generally to tea-incorporated and non-tobacco tea-like compositions for oral administration in dry form that mix with saliva to form a mixture that is intended, in part, to be safely swallowed and digested by a consumer. More particularly, the disclosure pertains to enhanced embodiments of the tea-like compositions of the type disclosed in the aforesaid commonly owned U.S. patent application Ser. No. 15/100,288 (published as US20160324777).

### Background Art

The compositions disclosed in the aforesaid US20160324777 (Victor et al) application can be consumed and enjoyed as nutritional and energy producing products and are comprised of green tea and/or other leaf material and one or more additional ingredients which, along with the manner in which the composition is consumed and the material of the pouch in which the compositions are contained, differentiate the product from common tea leaves in a bag intended for submersion into water. One embodiment includes a particlized tea based formulation comprising one or more of the following constituents: plant leaves and/or flowers and/or stems and/or seeds and/or roots; a stimulant and/or caffeine; one or more humectants; salt (NaCl); water; and additional plant material and/or cellulose. In a preferred embodiment the plant material is a green tea. A preferred oral delivery method for the tea or tea-like composition employs a small liquid-porous webbed pouch configured to be placed inside the consumer's mouth between the cheek and gum where saliva can pervade the pouch and distribute the resulting composition-saliva liquid mix in the user's mouth. The plant material in the composition is particlized (i.e., torn, chopped, shredded, etc.) or otherwise provided in small pieces but not so small as to fit through interstices in the webbed pouch material while the smaller, dissolvable non-plant material particles are able to fit through the interstices in the webbed pouch material when mixed with saliva. unless fully or partially dissolved in the saliva mixture. In another embodiment, the tea or tea-like composition may be packaged as loose particles inside a tin or other container so as to be administered as pinch of a chewable product. In either case, the resulting product is a tea product that: (a) can be enjoyed over time as the composition soaks in saliva; (b) is organic and has health benefits for the user; (c) has a desirable taste; (d) does not require refrigeration for storage; and (e) is not readily susceptible to growth of mold and unhealthy bacteria.

It would be desirable to further enhance the enjoyment of such compositions by providing a positive tactile sensation to the tongue and mouth along with the desirable taste. In this regard, it is known to provide a pleasant popping sensation in a user's mouth by adding appropriate content to candy and medications. Medications? For example, there are candies that are made by mixing certain ingredients and heating them until they melt into a syrup, then exposing the mixture to pressurized carbon dioxide (CO₂) gas at about six hundred pounds per square inch and allowing it to cool. The process causes tiny internally pressurized bubbles to be trapped inside the candy. When the candy is placed in a consumer's mouth and comes into contact with saliva, the candy breaks and dissolves, releasing the carbon dioxide from the bubbles, resulting in a popping and sizzling sound and leaving a tingling sensation on the consumer's tongue and other soft tissue in the user's mouth. Variations of that process and other techniques for treating oral consumables to produce the same type of sensation in a user's oral cavity may be found in, for example, U.S. Pat. No. 3,012,893 (Kremzner et al), U.S. Pat. No. 4,289,794 (Kleiner et al), U.S. Pat. No. 4,837,039 (Gallart et al), U.S. Pat. No. 6,479,085 (Archibald) and US20040131743 (Ajao et al), the entire disclosures in which are incorporated herein by reference. Therefore, in light of the above, and for other reasons that become apparent from the disclosure below, it is an object of the present disclosure to provide the same or a similar sensation for users of tea-like products of the type described above.

### SUMMARY OF THE DISCLOSURE

This Summary is provided to introduce concepts in a simplified form that are further described below in the Detailed Description. This Summary of itself is not intended to identify key features or essential features of the claimed subject matter, nor is it intended as an aid in determining the scope of the claimed subject matter.

An aspect of the disclosure pertains to a non-tobacco formulation product for oral administration to a user comprising pieces or particles of a non-tobacco tea-like composition having a water content of three percent or less by weight and comprising plant material, an additive mixed with the composition and responsive to being wetted by the user's saliva to cause gaseous bubbles to be released and provide a popping or cracking effect in the user's mouth, and a liquid-porous pouch-type container enclosing the composition and additive therein while permitting liquid to pass therethrough. The additive may have gas bubbles entrapped therein that are released when the additive is wetted. In a preferred embodiment the additive comprises a CO₂-infused sugar or sugar substitute based solid, and may further include at least one of sodium bicarbonate and tartaric acid. The formulation may further include medium-chain triglyceride (MCT) oil as a particle binder.

Another aspect of the disclosure pertains to a non-tobacco formulation product for oral administration in dry form to a user comprising a non-tobacco tea-like composition having a water content of three present or less by weight and comprising plant material, a liquid-porous container having walls containing the composition while permitting liquid to pass therethrough, and an additive coating on said container walls responsive to being wetted by the user's saliva to cause gaseous bubbles to be released and provide a popping or cracking sensation in the user's mouth. The additive may have gas bubbles entrapped therein that are released when the additive is wetted. In a preferred embodiment the additive comprises a CO₂-infused sugar or sugar substitute based solid, and may further include at least one of sodium bicarbonate and tartaric acid. The formulation may further include medium-chain triglyceride (MCT) oil as a particle binder.

In accordance with still another aspect of the disclosure a particlized tea-like composition is provided with a bubble-containing additive that is responsive to saliva to cause the gaseous bubbles to release and provide a popping or cracking sensation in the user's mouth. For purposes of the present disclosure a preferred technique for achieving that sensation is the use of liquid activated carbon dioxide-infused sugar or sugar substitute based solids as a bubble-containing additive; in an alternative technique the additive may be a liquid activated sugar or sugar substitute in combination with sodium bicarbonate and tartaric acid. The additive is combined with the tea-like compositions disclosed in aforementioned US Pub. No. 2016/0324777 (Victor et al). Importantly, the resulting tea-like composition contains less than three percent water by weight. In some embodiments of the disclosure the composition may be coated with a sufficient amount of an edible fat and/or oil to maintain the liquid-activated products in solid form until used.

In another aspect the present disclosure relates to a formulation which may contain green tea and/or other tea or leaf ingredients such as, by way of example, yerba mate or peppermint leaf, and further contains the bubble-containing additive and such other ingredients as disclosed in aforementioned US20160324777 (Victor et al), but importantly with a percentage of water of substantially zero or in any case not greater than 3% by weight. In one or more exemplary embodiments of the disclosure, the formulation may further contain medium-chain triglyceride (MCT) oil representing between 0% and 30% by weight of such formulation.

Another aspect the present disclosure pertains to a method of utilizing the aforesaid composition in a pouch of the types disclosed in aforementioned US20160324777 (Victor et al) or other similar liquid-porous container wherein the formulation is not loose in the mouth. In one embodiment the pouch may be made of a natural and/or synthetic material that is porous to liquid to allow for the ingress and egress of saliva and/or other liquids. Alternatively, such formulation may be loose and delivered into the mouth for oral use without a pouch, akin to delivering a pinch of tobacco to the mouth as with oral tobacco products. Alternatively, the formulation may be separated whereby the bubble-containing additive is adhered to the outside of the liquid-porous pouch, the other elements of the formulation being contained within the pouch.

In other aspects, the present disclosure is directed to compositions and formulations for oral administration of green tea and/or other leaf material and an additive comprising saliva-activated CO₂-infused sugar or sugar substitute-based solids, or alternatively or cooperatively, liquid-activated, sugar or sugar substitute-based solids in combination with sodium bicarbonate and tartaric acid, and which may also contain medium-chain triglyceride (MCT) oil and such other ingredients as set forth in US20160324777 (Victor et al). In the present disclosure, CO₂ can be infused in the sugar or sugar substitute-based solids in any commercially feasible manner such as the use of high pressure described above and/or the methods described in the aforesaid Kremzner et al, Kleiner et al, Gallart et al, Archibald and (Ajao et al) patents.

### BRIEF DESCRIPTION OF THE DRAWINGS

By way of example, a specific embodiment of the disclosure will now be described, partially with reference to the accompanying drawings, in which:

FIG. 1A is a perspective view of a pouch for containing the composition of the present disclosure.

FIG. 1B is a perspective view in longitudinal section of the pouch FIG. 1A showing the contained composition.

### DETAILED DESCRIPTION OF THE DISCLOSURE

Terminology

The following terms shall have the following meanings in the following disclosure and claims:

“Pouch” means a liquid-porous container configured for placement in the mouth of a user and to contain pieces of the compositions and formulations described herein while permitting those pieces to be wetted and/or soaked by saliva. The liquid porosity may be provided by the pouch material or, preferably, by providing the material as a web or screen with interstices for passing liquid therethrough.

“Particlized” means pieces of the compositions and formulations described herein, the pieces being large enough to not be able to pass through interstices of the pouch.

The embodiments in this disclosure are described more fully hereinafter, partially with reference to the accompanying drawings of the pouch. The subject matter of the disclosure may be embodied in many different forms and should not be construed as limited to the embodiments described specifically herein. Rather, these embodiments are provided so that this disclosure will be thorough and complete, and will fully convey the scope of the subject matter to those skilled in the art.

Unless otherwise indicated or made clear from the context in which it is described, alternative and optional constituents or combinations in any of the disclosed embodiments and examples are interchangeable with each other. That is, a component described in one embodiment or example should be understood to be interchangeable or substitutable for one or more corresponding but different components in another described example or embodiment and, likewise, an optional feature of one embodiment or example may optionally also be used in other embodiments and examples. More generally, the elements and features of any disclosed example or embodiment should be understood to be disclosed generally for use with other aspects and other examples and embodiments. A reference to a component, additive or ingredient being operative or configured to perform one or more specified functions or the like, or included for a particular purpose including flavoring or for volume in the product, or for effect of the ingredient, is intended to mean that it can perform such functions in at least certain embodiments, and may well be able to perform also one or more other functions and/or operations.

In a preferred embodiment of the present disclosure, a formulation includes an additive containing multiple gaseous bubbles and comprising liquid activated CO₂-infused sugar or sugar substitute-based solids as ingredients in an amount between 1% and 99% by weight of the formulation. Alternatively, the additive containing multiple gaseous bubbles includes liquid activated sugar or sugar substitute-based solids and sodium bicarbonate and tartaric acid as ingredients in an amount between 1% and 99% by weight of the formulation. The formulations are for oral administration and may additionally contain green tea and/or other teas or leaf ingredients, and medium-chain triglyceride (MCT) oil and such other ingredients as set forth in US20160324777 (Victor et al). The formulations may be provided as loose pieces in a container or, preferably, as pieces enclosed within a liquid-porous pouch or similar structure made of a natural and/or synthetic material. In addition to the formulations, the methods of making these formulations and the methods for oral use thereof are also aspects of the disclosure,

In other embodiments of the disclosure the liquid-activated CO₂-infused sugar or sugar substitute-based solids with or without flavoring and, alternatively, liquid-activated, sugar or sugar substitute-based solids further comprised of sodium bicarbonate and tartaric acid with or without flavoring, are substantially, or de facto, the only ingredients within the pouch or similar structure for oral administration.

In another exemplary embodiment of the present disclosure, the above described liquid activated additive with contained bubbles, with and/or without flavor, comprises between 0.1% and 99.0% of the weight of the composition with the additive adhered or otherwise fixed to the exterior of the pouch, or similar liquid pervious structure, and distinct from the formulation within the pouch or structure, which formulation may contain green tea and/or other leaf material, and may additionally contain a medium-chain triglyceride (MCT) oil, and such other ingredients as set forth in aforementioned US20160324777. The water content of the formulation is preferably substantially 0% and in any event less than 3% by weight. The pouch or similar structure is comprised of a natural and/or synthetic liquid-porous material.

Depending on the intensity of the tactile oral tingling sensation desired from a particular embodiment in which the additive is part of the composition, the additive may comprise anywhere from 0% to 99% by weight of the composition.

In another exemplary embodiment the composition may contain any of the aforesaid additives plus one or more of green tea and/or other tea or leaf ingredients, MCT oil, water-soluble extract of tetrahydrocannabinol (THC), water-soluble extract of cannabidiol (CBD), plant material harvested from Cannabis Sativa, and some or all of those ingredients set forth in US20160324777.

In another exemplary embodiment the composition may contain any of the aforesaid additives and further comprise one or more of the following: water soluble Tetrahydrocannbidiol extract, oil soluble Tetrahydrocannabidiol extract, Tetrahydrocannabidiol as existing in leaf or other form, water soluble Cannabidiol extract, oil soluble Cannabidiol extract, and/or Cannabidiol as existing in leaf or other form. The composition may additionally comprise green tea and/or other tea or leaf ingredients, MCT oil, plant material harvested from Cannabis Sativa, and some or all of those ingredients set forth in the embodiments and claims set forth in US20160324777.

Since the compositions of the present invention contain no more than 3% water and, optimally, substantially 0% water by weight, the formulations described herein are functionally, if not de facto, dry, in contrast to many tobacco pouch products that are intentionally provided moist to achieve their intended functions and purposes. The dry formulation used in the present disclosure permits the liquid-activated constituents described herein to remain dormant until activated by saliva in a user's mouth.

The MCT oil, when used in some disclosed embodiments, represents up to 30% by weight of the formulations and serves as a binder for the small particle dry ingredients because: (1) in the manufacturing process, as the dry constituents are injected, deposited, released or otherwise inserted into a pouch or other similar structure, the smaller particle dry ingredients can become aerosolized and result in problems with equipment, labor and quality control; and 2) water is an undesirable ingredient in the formulations of the disclosure because a high water activity level can cause the formation of, and support the existence of, yeast, mold and bacteria. Other liquids, like ethyl alcohol, which might not cause health risks of water and which might evaporate quickly, are undesirable in that they are combustible.

Liquid-activated CO₂-infused, sugar or sugar substitute-based solids, or, alternatively, liquid-activated, sugar or sugar substitute-based solids further comprised of sodium bicarbonate and tartaric acid, are typically and readily commercially available to retail consumers when further comprised of flavoring elements. When such aforementioned liquid-activated solid components are not further comprised of flavoring elements, the resulting components are not generally or desirably ingested by consumers.

Embodiments described herein, because they are formulated to be comprised of substantially 0% water or functionally, if not de facto dry or in any event less than 3% water, are well suited to a formulation with added elements comprising liquid-activated, CO₂-infused solids since those elements must not be exposed to moisture during formulation, processing or storage.

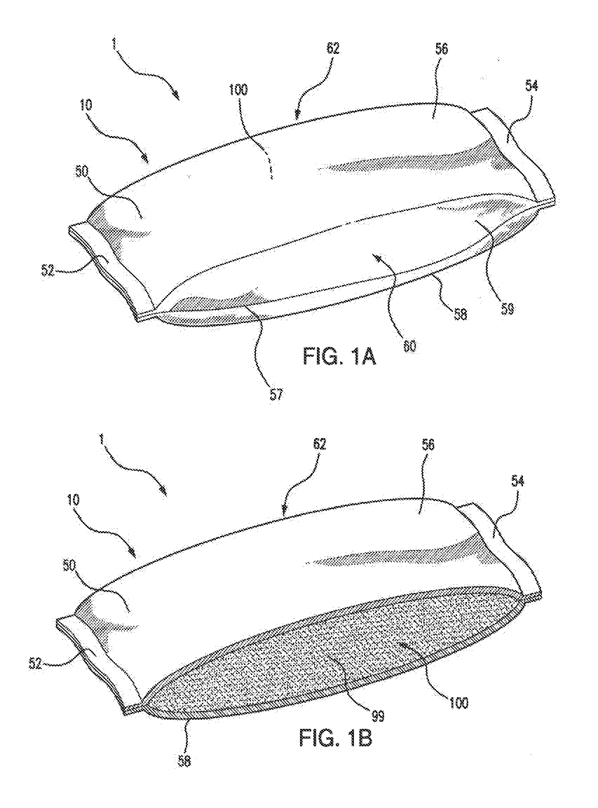

The pouch for containing the formulations of the preferred embodiments is porous to liquid and preferably made from a web material in which the interstices are small enough to permit saliva to pass through but large enough to prevent passage of the particlized components of the contained composition. FIG. 1A is a perspective view of an example of a filled pouch 1. The pouch may be filled with any of the compositions and additives 100 disclosed herein; alternatively, the pouch may be coated with an additive as described. The pouch 1 may have a first closed end 52 and a second closed end 54 and may be comprised of an upper pouch 56 and a lower pouch 58. A first side 60 has a first side wall 59 optionally having a first side pleat 57. A second side 62 of the pouch has a second side wall 63 optionally having a second side pleat 64. The example pouch of FIG. 1A is generally rectangular pouch but it will be understood that the pouch may have any configuration consistent with the functional requirements described herein. As noted, the pouch material 50 is in the form of a web. A cross-sectional view of pouch 1 filled with the composition materials 99 is shown in FIG. 1B.

It will be appreciated from the foregoing that the disclosure herein has the following features:

(a) a bubble-containing additive to a tea-like composition enclosed in a liquid-porous pouch, wherein the additive is responsive to saliva to cause the gaseous bubbles to release and provide a popping or cracking sensation in the user's mouth;

(b) a bubble-containing substance enclosed in a liquid-porous pouch, wherein the substance is responsive to saliva to cause the gaseous bubbles to release and provide a popping or cracking sensation in the user's mouth; and

(c) a bubble-containing additive coating a liquid-porous pouch that contains a tea-like composition, wherein the additive is responsive to saliva to cause the gaseous bubbles to release and provide a popping or cracking sensation in the user's mouth.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the described features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to reside within the scope of the present disclosure. Furthermore, although the present disclosure has been described herein in the context of particular formulations, those of ordinary skill in the art will recognize that its usefulness is not limited thereto and that the present disclosure may be beneficially implemented in substantially any number of formulations. Accordingly, the claims set forth below should be construed in view of the full breadth and spirit of the present disclosure as described herein.

What is claimed is:

1. A non-tobacco formulation product for oral administration in dry form to a user comprising;
a non-tobacco tea-like composition comprising solid plant material;
an additive mixed with said composition and responsive to being wetted by the user's saliva to cause gaseous bubbles to be released and provide a popping or cracking sensation in the user's mouth; and
a liquid-porous container enclosing the composition and additive therein while permitting liquid to pass therethrough;
wherein water content of said composition is between 0% and 3% by weight.

2. The non-tobacco formulation product of claim 1 wherein said additive has the gaseous bubbles entrapped therein.

3. The non-tobacco formulation product of claim 2 wherein said additive comprises a CO₂-infused sugar or sugar substitute based solid.

4. The non-tobacco formulation product of claim 2 wherein said additive comprises at least one of sodium bicarbonate and tartaric acid.

5. The non-tobacco formulation product of claim 2 further comprising medium-chain triglyceride (MCT) oil as a particle binder.

6. The non-tobacco formulation product of claim 2:
wherein said composition includes at least some particles that dissolve in saliva;
wherein said container is a pouch comprising mesh-like walls with interstices for passing saliva therethrough, the interstices being sufficiently small to block passage therethrough of the contained solid composition except for particles fully or partially dissolved in saliva.

7. The non-tobacco formulation product of claim 2 wherein said composition further includes some or all of the following constituents: green tea, MCT oil, water-soluble extract of tetrahydrocannabinol (THC), water-soluble extract of cannabidiol (CBD), plant material harvested from Cannabis Sativa and other tea or leaf ingredients.

8. The non-tobacco formulation product of claim 2 wherein said composition further includes some or all of the following constituents: water soluble Tetrahydrocannbidiol extract, oil soluble Tetrahydrocannabidiol extract, Tetrahydrocannabidiol, water soluble Cannabidiol extract, oil soluble Cannabidiol extract, and/or Cannabidiol.

9. A non-tobacco formulation for oral administration in dry form to a user comprising;
a non-tobacco tea-like composition comprising plant material;
a liquid-porous container having walls containing the composition while permitting liquid to pass therethrough; and
an additive coating said container walls and responsive to being wetted by the user's saliva to cause gaseous bubbles to be released and provide a popping or cracking sensation in the user's mouth.

10. The non-tobacco formulation product of claim 9 wherein said additive has the gaseous bubbles entrapped therein.

11. The non-tobacco formulation product of claim 10 wherein said additive comprises a CO₂-infused sugar or sugar substitute based solid.

12. The non-tobacco formulation product of claim 10 wherein said additive further comprises at least one of sodium bicarbonate and tartaric acid.

13. The non-tobacco formulation product of claim 10 further comprising medium-chain triglyceride (MCT) oil as a particle binder.

14. The non-tobacco formulation product of claim 10:
wherein said composition includes at least some particles that dissolve in saliva; and
wherein said container is a pouch comprising mesh-like walls with interstices for passing liquid therethrough, the interstices being sufficiently small to block passage therethrough of the contained solid composition except for particles fully or partially dissolved in saliva.

15. The non-tobacco formulation product of claim 10 wherein said composition further includes some or all of the following constituents: green tea, MCT oil, water-soluble extract of tetrahydrocannabinol (THC), water-soluble extract of cannabidiol (CBD), plant material harvested from Cannabis Sativa and other tea or leaf ingredients.

16. The non-tobacco formulation product of claim 10 wherein said composition further includes some or all of the following constituents: water soluble Tetra hydrocannbidiol extract, oil soluble Tetrahydrocannabidiol extract, Tetrahydrocannabidiol, water soluble Cannabidiol extract, oil soluble Cannabidiol extract, and/or Cannabidiol.

17. A non-tobacco formulation comprising:
a non-tobacco composition comprising green tea;
an additive mixed with said composition and responsive to being wetted by the user's saliva to cause gaseous bubbles to be released and provide a popping or cracking sensation in the user's mouth;
wherein water content of said composition is between 0% and 3% by weight.

18. The non-tobacco formulation of claim 17 wherein said additive has the gaseous bubbles entrapped therein and comprises a CO₂-infused sugar or sugar substitute based solid said additive.

19. The non-tobacco formulation of claim 18 further comprising medium-chain triglyceride (MCT) oil as a particle binder.

20. A product including the non-tobacco formulation of claim 17 further comprising:
a liquid-porous container having walls containing the composition while permitting liquid to pass therethrough;
wherein said container is a pouch comprising mesh-like walls with interstices for passing saliva therethrough, the interstices being sufficiently small to block passage therethrough of the contained solid composition except for particles fully or partially dissolved in saliva.
